(19) United States
(12) Patent Application Publication
(10) Pub. No.: US 2019/0046011 A1
(43) Pub. Date: Feb. 14, 2019

(54) FLEXIBLE TUBE INSERTION APPARATUS

(71) Applicant: OLYMPUS CORPORATION, Tokyo (JP)

(72) Inventor: Yuichi IKEDA, Tama-shi (JP)

(73) Assignee: OLYMPUS CORPORATION, Tokyo (JP)

(21) Appl. No.: 16/159,775

(22) Filed: Oct. 15, 2018

Related U.S. Application Data
(63) Continuation of application No. PCT/JP2016/062813, filed on Apr. 22, 2016.

(51) Int. Cl. A61B 1/00; A61B 1/005

(52) U.S. Cl. CPC A61B 1/00078; A61B 1/00147; A61M 25/0053; A61B 1/00165; A61B 1/0051

(57) ABSTRACT

A flexible tube insertion apparatus includes a flexible tube segmented into segments aligned its central axis and to be inserted into a target body, a variable stiffness portion to vary a bending stiffness of the tube, a detection sensor to detect state information on the tube, a calculator to calculate shape information on the tube based on the state information, and a controller to control a change of the stiffness implemented by the variable stiffness portion. The controller controls the change of the stiffness to a segment corresponding to a displacement portion of an insertion path, in accordance with a displacement quantity of a second path of the tube based on the shape information calculated at a second time, with respect to a first path of the tube based on the shape information calculated at a first time.

### CROSS-REFERENCE TO RELATED APPLICATIONS

This application is a Continuation Application of PCT Application No. PCT/JP2016/062813, filed Apr. 22, 2016, the entire contents of which are incorporated herein by reference.

### BACKGROUND OF THE INVENTION

### 1. Field of the Invention

The present invention relates to a flexible tube insertion apparatus that inserts a flexible tube into a tract section of an insertion target body.

### 2. Description of the Related Art

A flexible tube of an insertion section of an endoscope apparatus disclosed in, for example, Jpn. Pat. Appln. KOKAI Publication No. 2016-7434 is partitioned in segments aligned in a row along a central axis of the insertion section. The endoscope apparatus changes a bending stiffness of the flexible tube to a bending stiffness suitable for insertion by units of segments in accordance with the shape of the flexible tube detected by a shape detection sensor. This allows insertability of the insertion section to improve when performing a push operation of the insertion section into a deep part of a tract section (for example, an intestine tract of a large intestine) of an insertion target body (for example, a large intestine).

A tube-like insertion apparatus disclosed in Jpn. Pat. Appln. KOKAI Publication No. 2015-16365, for example, provides an operator with control assistance information that is information necessary for insertion of an insertion section and that includes shape information regarding a shape of the insertion section and external force information regarding external force received by the insertion section. The operator is assisted on the insertion operation of the insertion section by the control assistance information.

### BRIEF SUMMARY OF THE INVENTION

A flexible tube insertion apparatus according to an aspect of the present invention includes: a flexible tube that is segmented into segments aligned in a row along a central axis of the flexible tube, and is to be inserted into a insertion target body; a variable stiffness portion that varies a bending stiffness of the flexible tube by units of the segments; a state detection sensor that detects state information regarding a state of the flexible tube; a shape calculator that calculates shape information regarding a shape of the flexible tube based on the state information; and a stiffness controller that controls a change of the bending stiffness implemented by the variable stiffness portion. The stiffness controller controls the change of the bending stiffness implemented by the variable stiffness portion with respect to a segment corresponding to a displacement portion of an insertion path, in accordance with a displacement quantity of a second insertion path of the flexible tube based on the shape information calculated by the shape calculator at a second time, with respect to a first insertion path of the flexible tube based on the shape information calculated by the shape calculator at a first time.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 is a schematic view of a flexible tube insertion apparatus according to an embodiment of the present invention.

FIG. 2 is a diagram explaining relationships among segments, a state detection sensor, a shape calculator, a stiffness controller, and variable stiffness portions.

FIG. 3 is a diagram explaining a change quantity of a bending stiffness in accordance with a displacement quantity of a second insertion path with respect to a first insertion path, and a target bending stiffness.

### DETAILED DESCRIPTION OF THE INVENTION

In the following, an embodiment of the present invention will be explained in detail with reference to the figures. In some of the figures, illustrations of some members are omitted to obtain a clarified illustration.

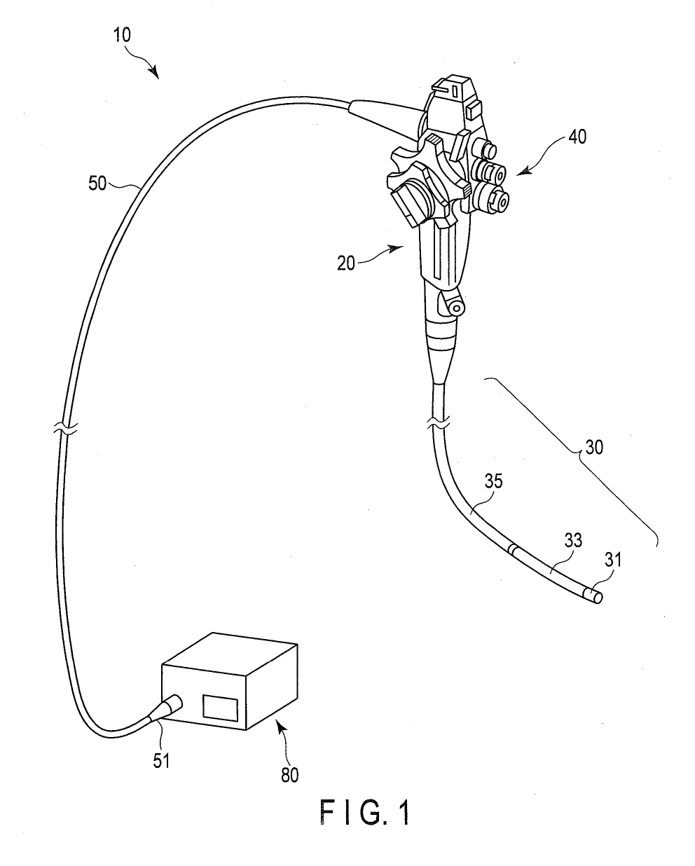

As shown in FIG. 1, a flexible tube insertion apparatus (hereinafter referred to as an insertion apparatus 10 ) comprises an endoscope 20 and a control device 80 that controls the endoscope 20. The control device 80 functions, for example, as a stiffness control apparatus that controls a bending stiffness of a flexible tube 35 of an insertion section 30 that is arranged in the endoscope 20. Although not illustrated, the insertion apparatus 10 may also comprise a display device that displays an image imaged by the endoscope 20, and a light source device that emits light for the endoscope 20 to observe and image.

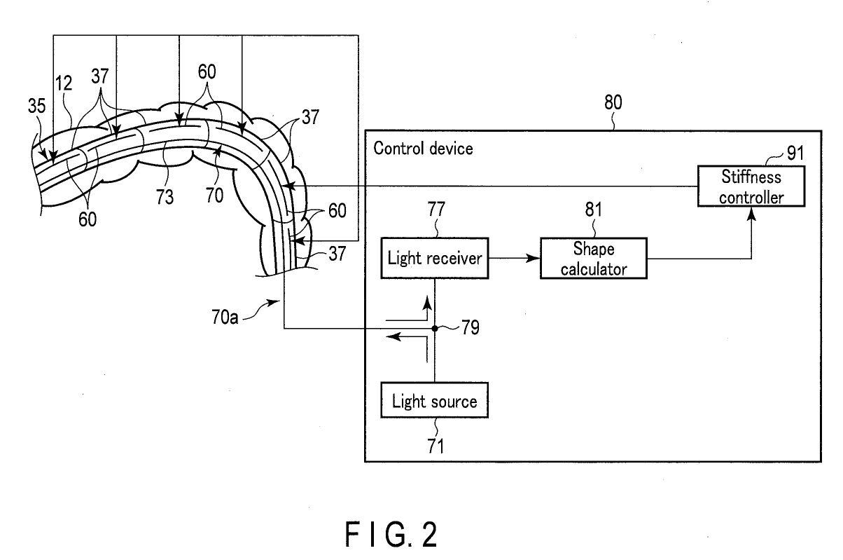

The endoscope 20 will be explained as, for example, a medical soft endoscope; however, the endoscope 20 is not limited to this. The endoscope 20 only has to include a soft insertion section 30 that is to be inserted in a tract section (for example, an intestine tract 12 of a large intestine (refer to FIG. 2)) of an insertion target body (for example, a patient), such as an industrial soft endoscope, a catheter, and a treatment device. The insertion section 30 only has to include a portion (for example, a flexible tube 35 explained later on) with flexibility that can be bent by external force. The endoscope 20 may be a direct-view type endoscope, or a side-view type endoscope. The insertion target body, for example, is not limited to a human, and may be an animal, or other structures. The tract section may be, for example, an industrial pipe.

The endoscope 20 comprises the insertion section 30, a control section 40 that is connected to a proximal end of the insertion section 30 and operates the endoscope 20, and a universal cord 50 that extends from a side surface of the control section 40. The universal cord 50 has a connector 51 that is attachable to and detachable from the control device 80.

The insertion section 30 is tubular, elongated, and flexible. The insertion section 30 moves back and forth inside the tract section with respect to the tract section. The insertion section 30 is bendable in accordance with a shape of the tract section. The insertion section 30 comprises, from its distal end to its proximal end, a distal hard section 31, a bendable section 33, and the flexible tube 35, in this order. The distal hard section 31 and the bendable section 33 are shorter than the flexible tube 35. Therefore, in the present embodiment, the distal hard section 31, the bendable section 33, and the distal end of the flexible tube 35 are regarded as the distal end of the insertion section 30. The flexible tube 35 has flexibility and is bent by external force.

As shown in FIG. 2, the flexible tube 35 of the insertion section 30 is segmented into segments 37 aligned in a row along a central axis of the insertion section 30. For example, the segments 37 are present over an entire length of the flexible tube 35. The bending stiffness of each segment 37 is independently changeable by the control of the control device 80. Accordingly, the bending stiffness of the flexible tube 35 is partially changeable by the bending stiffness of each segment 37 that is independently controlled by the control device 80. Each segment 37 may function as a virtual region that does not actually exist, or may function as a structure that actually exists. Each length of the segments 37 may be identical to or may differ from each other. For example, the length of a portion to be inserted into the insertion target body at the insertion section 30 depends on the insertion target body. Accordingly, the portion to be inserted may be considered as being segmented into segments 37, and a portion that is arranged outside the insertion target body and is not to be inserted into the insertion target body may be considered as being one segment 37.

The insertion apparatus 10 includes variable stiffness portions 60 that each have stiffness that is variable by the control of the control device 80, and that change the bending stiffness of the flexible tube 35 by the stiffness. In the present embodiment, the variable stiffness portions 60 vary the bending stiffness of the flexible tube 35 in the insertion section 30 by units of segments 37. Therefore, it is explained that, for example, each variable stiffness portion 60 is embedded in each segment 37, and the variable stiffness portions 60 are embedded over the entire length of the flexible tube 35. The variable stiffness portions 60 only have to be arranged on portions that are inserted in the tract section and that need to have the bending stiffness changed in the flexible tube 35. That is, the variable stiffness portions 60 may be embedded only in some of the segments 37.

Portions on which the variable stiffness portions 60 are provided may at least function as the segments 37. A variable stiffness portion 60 may be embedded over segments 37. The variable stiffness portions 60 may be aligned in a row, or in rows along a central axis of the insertion section 30. In a case where the variable stiffness portions 60 are aligned in rows, a set of variable stiffness portions 60 may be provided at the same position so that the variable stiffness portions 60 are adjacent to each other in a circumferential direction of the flexible tube 35 or so that the variable stiffness portions 60 are shifted along the central axis of the insertion section 30.

Although not shown, each variable stiffness portion 60 is configured by an actuator that comprises, for example, a coil pipe that is formed by a metal wire, and an electroactive polymer artificial muscle (hereinafter referred to as EPAM) encapsulated inside the coil pipe. The central axis of the coil pipe is coincide with or in parallel to the central axis of the insertion section 30. The coil pipe includes a pair of electrodes that is provided on the both ends of the coil pipe.

Electrodes of each variable stiffness portion 60 are connected to the control device 80 through signal cables that are embedded in the endoscope 20, respectively, and receive electric power from the control device 80. When a voltage is applied to the EPAM through the electrode, the EPAM attempts to expand and contract along the central axis of the coil pipe. However, the coil pipe restricts the expansion and contraction of the EPAM. In this manner, the stiffness of a variable stiffness portion 60 changes. The stiffness of the variable stiffness portion 60 increases as the applied voltage value increases. The stiffness of the variable stiffness portion 60 changes; the bending stiffness of the corresponding segment 37 also changes. The electric power is supplied independently to each pair of electrodes of the variable stiffness portions 60. Therefore, the stiffness of each variable stiffness portion 60 changes independently; the bending stiffness of each segment 37 also changes independently. In this manner, each variable stiffness portion 60 causes the bending stiffness of the corresponding segment 37 to change according to the stiffness change of the variable stiffness portion 60; the bending stiffness change of the segment 37 causes the bending stiffness of the flexible tube 35 to partially change.

The variable stiffness portion 60 may also use a shape-memory alloy instead of the EPAM.

The insertion apparatus 10 includes a state detection sensor 70 that detects state information of the flexible tube 35 regarding the state of the flexible tube 35. In the present embodiment, the state of the flexible tube 35 indicates a bending state of the flexible tube 35, for example, a bending quantity (magnitude of bending) of the flexible tube 35. The state of the flexible tube 35 may include a bending direction of the flexible tube 35.

As an example, the state detection sensor 70 includes a fiber sensor 70a that utilizes a loss of a light transmission quantity caused by bending an optical fiber 73. The fiber sensor 70 a comprises a light source 71 that emits light, the optical fiber 73 that guides the light, a reflector (not shown) that reflects light so that the light guided by the optical fiber 73 travels backwards in the optical fiber 73, a light receiver 77 that receives the reflected light, and a light branching unit 79. The light source 71 includes, for example, an LED. The light source 71 is separate from a light source of the light source device that emits light for observation and imaging. The optical fiber 73 is embedded in the endoscope 20 and has flexibility. The optical fiber 73 has detection targets (not shown) that are mounted on the insertion section 30. The detection targets are arranged at positions that are different from each other along a longitudinal axial of the optical fiber 73. The detection targets only have to be arranged at portions where the bending stiffness of the flexible tube 35 is to be changed. Accordingly, in the present embodiment, suppose the detection targets are arranged on each segment 37 inside the flexible tube 35. The optical fiber 73 is arranged alongside the variable stiffness portions 60 in the flexible tube 35. The reflector is arranged at the distal end of the optical fiber 73, which is positioned at the distal end of the insertion section 30. The light receiver 77 may comprise, for example, an element for spectral dispersion such as a spectroscope or a color filter, and a light receiving element such as a photodiode. The light source 71, the light receiver 77, and the proximal end of the optical fiber 73 are optically connected to the light branching unit 79. The light branching unit 79 comprises, for example, an optical coupler or a half mirror. The light branching unit 79 guides the light emitted from the light source 71 to the optical fiber 73, and guides returned light reflected by the reflector and guided by the optical fiber 73 to the light receiver 77. That is, the light travels in the order of the light source 71, the light branching unit 79, the optical fiber 73, the reflector, the optical fiber 73, the light branching unit 79, and the light receiver 77. The light source 71, the light receiver 77, and the light branching unit 79 are, for example, mounted on the control device 80.

When the insertion section 30 is bent, the optical fiber 73 is bent in accordance with such bending. Accordingly, a part of light that propagates through the optical fiber 73 is emitted (leaks) outside through, for example, the detection targets that have sensitivity in different wavelengths from each other. The detection target changes optical characteristics of the optical fiber 73; such as the light transmission quantity of light of a predetermined wavelength. Therefore, when the optical fiber 73 is bent, the light transmission quantity of the optical fiber 73 changes in accordance with the bending quantity of the optical fiber 73. A light signal that includes information of the change in the light transmission quantity is received at the light receiver 77. The light receiver 77 outputs the light signal as state information of the flexible tube 35 to a shape calculator 81 explained later on, arranged in the control device 80.

One detection target may be arranged on one optical fiber 73; in which case optical fibers will be arranged. Furthermore, suppose detection targets are arranged at the same position or at nearby positions along the longitudinal axial of the optical fiber, and at positions different from each other in a circumferential direction about the longitudinal axis. In such case, the bending quantity and the bending direction can be detected by a combination of the detection results of the detection targets.

The state detection sensor 70 is not limited to comprising the fiber sensor 70a. The state detection sensor 70 may also comprise one of, for example, a strain sensor, an acceleration sensor, a gyro sensor, and an element such as a coil. The strain sensor detects, for example, a bending strain caused by an external force (pressure) that the flexible tube 35 receives externally (for example, from the tract section). The acceleration sensor detects accelerated velocity of the flexible tube 35. The gyro sensor detects angular velocity of the flexible tube 35. The element generates a magnetic field corresponding to the state of the flexible tube 35, such as the shape of the flexible tube 35.

The state detection sensor 70 constantly performs detection (operates) after a detection start instruction is input to the state detection sensor 70 from an input device (not shown). The timing of detection may be implemented every lapse of a certain time, which is not limited in particular. The input device is general equipment for input, which, for example, may be a keyboard, a pointing device such as a mouse, a tag reader, a button switch, a slider, or a dial. The input device is connected to the control device 80. The input device may be used to input various instructions for a user to operate the insertion apparatus 10.

The insertion apparatus 10 comprises the shape calculator 81 and a stiffness controller 91 that are arranged in the control device 80. The shape calculator 81 and the stiffness controller 91 are, for example, configured by a hardware circuit that includes ASIC, etc. The shape calculator 81 and the stiffness controller 91 may also be configured by a processor. In the case where the shape calculator 81 and the stiffness controller 91 are configured by a processor, a program code that causes the processor to function as the shape calculator 81 and the stiffness controller 91 by executing the processor has been stored in an internal memory or an external memory (not shown) that is accessible by the processor.

The shape calculator 81 calculates shape information regarding the shape of the flexible tube 35 along the central axis of the flexible tube 35 based on the state information. The shape calculator 81 calculates the shape information at a predetermined time. For example, the shape calculator 81 calculates the shape information of the flexible tube 35 from the relationship of characteristics between incoming light and outgoing light of the optical fiber 73. In detail, the shape calculator 81 calculates the shape information, specifically, the bending shape of the flexible tube 35 of a part that is actually bending, based on the state information output from the fiber sensor 70a. The bending shape of the flexible tube 35 includes, for example, a curvature radius of the flexible tube 35. The shape calculator 81 regards the bending shape of the flexible tube 35 that is calculated at each predetermined time as an insertion path of the flexible tube 35 in an insertion process. In the above manner, the shape calculator 81 calculates the insertion path of the flexible tube 35 at a predetermined time based on the state information of the flexible tube 35 detected by the state detection sensor 70 at a predetermined time. The shape calculator 81 calculates the shape information (insertion path) of each segment 37 based on the state information. The shape calculator 81 calculates the shape information of the flexible tube 35 by joining the shape information of each segment. The shape calculator 81 calculates the insertion path for each time.

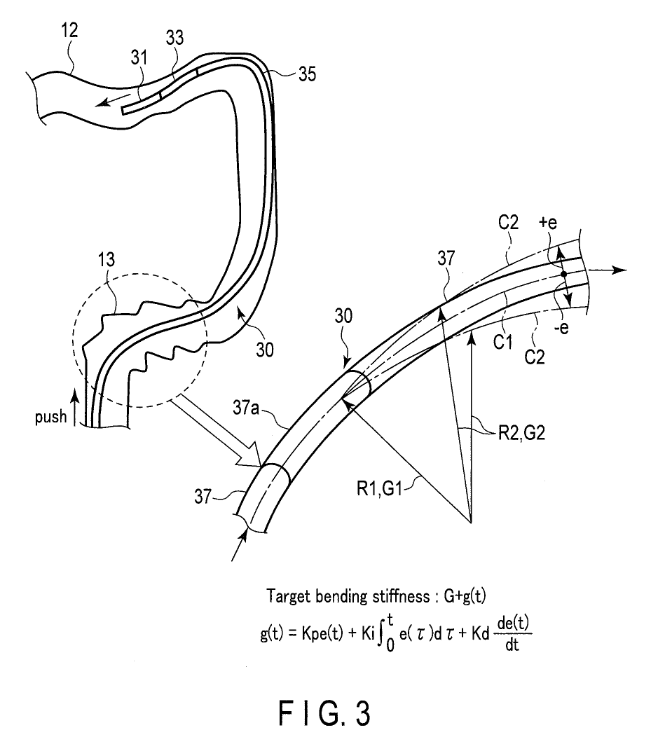

Here, the shape information calculated by the shape calculator 81 at first and second times will be referred to as first and second shape information. The second time is a time that is later than the first time. The insertion paths of the first and second times are referred to as first and second insertion paths C1 and C2 (see FIG. 3).

Furthermore, the shape calculator 81 also calculates information regarding a shape change of the segment 37 between the first time and the second time as the shape information based on the first shape information and the second shape information. Since the shape information indicates the insertion path of the flexible tube 35, the shape change indicates a displacement quantity ±e (see FIG. 3) of the second insertion path C2 with respect to the first insertion path C1.

Here, each segment 37 includes a front part that is arranged at the distal end of the segment 37, and indicates a front part in the insertion direction of the insertion section 30. For example, the shape calculator 81 calculates the shape information of the front part (insertion paths C1 and C2 and displacement quantity ±e of the insertion path).

As will be explained later on, the shape calculator 81 determines a change quantity g of the bending stiffness in accordance with the calculated displacement quantity ±e of the insertion path, and outputs it to the stiffness controller 91.

The stiffness controller 91 controls a change of the bending stiffness implemented by the variable stiffness portions 60 based on the change quantity g of the bending stiffness.

Here, with reference to FIG. 3, a process that determines the change quantity g in accordance with the displacement quantity ±e, and a process that determines a suitable bending stiffness based on the change quantity g, will be explained for one segment 37a. A suitable bending stiffness referred to herein indicates a bending stiffness (G+g(t)) to be a target value. The same process will also be implemented for each segment 37.

Here, the curvature radius R of the segment 37a and the bending stiffness G of the segment 37a at the first and second times will be referred to as curvature radiuses R1 and R2 and bending rigidities G1 and G2, respectively.

In the present embodiment, for example, in the case where the curvature radius R2 has changed less than the curvature radius R1 by a displacement quantity −e, the stiffness controller 91 controls the bending stiffness G so that the bending stiffness G2 becomes higher than the bending stiffness G1. That is, the stiffness controller 91 hardens the segment 37a so that the displacement quantity −e becomes 0, and the second insertion path C2 at the second time matches the first insertion path C1 at the first time.

In the present embodiment, in the case where, for example, the curvature radius R2 has changed more than the curvature radius R1 by a displacement quantity +e, the stiffness controller 91 controls the bending stiffness G so that the bending stiffness G2 becomes lower than the bending stiffness G1. That is, the stiffness controller 91 softens the segment 37a so that the displacement quantity +e becomes 0, and the second insertion path C2 at the second time matches the first insertion path C1 at the first time.

In this manner, in order to control the bending stiffness G at the stiffness controller 91, the shape calculator 81 determines the change quantity g of the bending stiffness G in accordance with the calculated displacement quantity ±e in the insertion paths C1 and C2 so that the original insertion path C1 (at the first time) is always maintained at any time, in other words, so that the displacement quantity ±e of the curvature radius R is 0. The change quantity g indicates, for example, a change quantity of the bending stiffness G2 with respect to the bending stiffness G1. Accordingly, for example, the bending stiffness (G+g(t)) that is the target value at the second time is a value obtained by adding the change quantity g to the bending stiffness G1.

The shape calculator 81 calculates the change quantity g by the following equation (1). Equation (1) indicates proportional control.

g(t)=Kpe(t) Equation (1)

“T” stands for time, and “Kp” is a constant number. In equation (1), the shape calculator 81 determines the change quantity g of the bending stiffness from time displacement of the second insertion path with respect to the first insertion path.

Here, in equation (1), the change quantity g is determined in proportion to the displacement quantity e. Since “Kp” is a constant number, as the displacement quantity e increases, the change quantity g also increases. That is, a problem arises in that the bending stiffness at the second time takes time to reach the bending stiffness (G+g(t)) of the target value. However, it is also difficult to determine the value of the constant number Kp in accordance with the displacement quantity e.

In order to solve this problem, the shape calculator 81 may also calculate the change quantity g by the following equation (2). Equation (2) indicates a PI control in which a time integral term of displacement is added to equation (1).

g(t)=Kpe(t)+Ki∫₀¹e(τ)dτ Equation (2)

“Ki” is a constant number. By equation (2), the shape calculator 81 determines the change quantity g of the bending stiffness from time integral with respect to the displacement of the second insertion path with respect to the first insertion path.

When a state of displacement, which is a deviation, continues for a long time, equation (2), to which the time integral term is added, serves the role of trying to increase the change quantity g accordingly so as to rapidly approximate the bending stiffness to the bending stiffness (G+g(t)), which is the target value.

In the PI control, the displacement is reformed more promptly as the integral time becomes smaller. However, as the integral time becomes smaller, an overshoot may occur, in which the bending stiffness exceeds the bending stiffness (G+g(t)) of the target value, or a vibration hunting may occur about the bending stiffness (G+g(t)), which is a target value.

In order to solve this problem, the shape calculator 81 may also calculate the change quantity g by the following equation (3). Equation (3) indicates a PID control in which a time differential term of displacement is added to equation (2).

$$g ( t ) = Kpe ( t ) + Ki {\int }_{0}^{t} e ( τ ) d τ + Kd \frac{de ( t )}{dt} \qquad Equation ( 3 )$$

“Kd” is a constant number. The shape calculator 81 determines the change quantity g of the bending stiffness from time differential with respect to the displacement of the second insertion path with respect to the first insertion path.

In the case where the change quantity g drastically changes, the time differential term has a damping effect that attempts to suppress drastic changes in the bending stiffness that is proportional to the magnitude of the change quantity g.

In this manner, it is desirable for the change quantity g to be determined based on the time displacement, the time integral with respect to the displacement, and the time differential with respect to the displacement.

The shape calculator 81 determines the change quantity g in accordance with the shape information (displacement quantity e), and outputs the determined change quantity g to the stiffness controller 91. The stiffness controller 91 controls the bending stiffness G based on the change quantity g.

For example, suppose the shape calculator 81 calculates the displacement quantity −e at the second time. In this state, when the insertion force quantity is added to the insertion section 30, the bending stiffness G2 here may fail to convert the insertion force quantity to a driving force that drives the insertion section 30, and may cause the flexible tube 35 to buckle. Accordingly, this may cause the insertability of the insertion section 30 that includes the flexible tube 35 to decrease. In the present embodiment, the shape calculator 81 determines a change quantity gA in accordance with the displacement quantity −e, and the stiffness controller 91 controls the bending stiffness G2 to become higher than the bending stiffness G1 based on the change quantity gA, so as to cause the second insertion path C2 to match the first insertion path C1. Therefore, the insertion force quantity is utilized as the driving force, and the flexible tube 35 passes through the curved portion 13 without buckling, which, without departing from the original insertion path C1, improves the insertability of the insertion section 30.

For example, suppose the shape calculator 81 calculates the displacement quantity +e at the second time. In this state, when the insertion force quantity is added to the insertion section 30, the bending stiffness G2 here may cause the insertion force quantity to be converted to a force that pushes up, for example, the large intestine wall of the large intestine. In this manner, the large intestine wall will be pushed up, and the insertion section 30 will unintentionally apply an excessive load to the large intestine wall, which will cause pain to the patient. In the present embodiment, the shape calculator 81 determines a change quantity gB in accordance with the displacement quantity +e, and the stiffness controller 91 controls the bending stiffness G2 to become lower than the bending stiffness G1 based on the change quantity gB, so as to cause the second insertion path C2 to match the first insertion path C1. Therefore, even if the insertion force quantity is added to the insertion section 30, the insertion force quantity will be utilized as the driving force, and the insertion section 30 will be driven without applying an excessive load to the large intestine wall. Accordingly, the insertion section 30 would not unintentionally apply an excessive load to the large intestine wall, which would cause less pain to the patient.

In this manner, the stiffness controller 91 controls the change of the bending stiffness implemented by the variable stiffness portion 60 with respect to a segment 37 corresponding to the displacement portion of the insertion path, in accordance with the displacement quantity of the second insertion path C2 of the flexible tube 35 in the insertion section 30 based on the second shape information calculated by the shape calculator 81 at the second time with respect to the first insertion path C1 of the flexible tube 35 in the insertion section 30 based on the first shape information calculated by the shape calculator 81 at the first time. The bending stiffness after the change refers to a bending stiffness that provides, to each segment 37 through the variable stiffness portion 60, a bending stiffness distribution suitable for insertion of the flexible tube 35 in accordance with the shape information. The bending stiffness refers to a bending stiffness that provides the stiffness distribution to the flexible tube 35 through the segment 37.

Accordingly, even if the insertion force quantity is added to the insertion section 30, the insertion force quantity would not be converted to a force that pushes up, for example, the large intestine wall of the large intestine, and would instead be utilized as the driving force to drive the insertion section 30. Therefore, the flexible tube 35 passes through the curved portion 13 without buckling, which, without departing from the original insertion path, improves the insertability of the insertion section 30. Furthermore, the insertion force quantity would not cause the large intestine wall to be pushed up. Therefore, the insertion section 30 would not unintentionally apply an excessive load to the large intestine wall, which would cause the patient less pain.

In the present embodiment, the stiffness controller 91 controls the bending stiffness in accordance with the displacement quantity ±e of the second insertion path C2 with respect to the first insertion path C1. Accordingly, in the present embodiment, the flexible tube 35 can be prevented from buckling, insertability to a deep part of the tract section can be improved, and the load applied to the insertion target body can be reduced without an excessive load being unintentionally applied to the wall portion of the tract section.

In the present embodiment, in each segment 37, the bending stiffness can be controlled. Accordingly, in the present embodiment, the bending stiffness of the flexible tube 35 can be finely controlled.

In the present embodiment, the shape calculator 81 determines the change quantity g of the bending stiffness in accordance with the displacement quantity e. The stiffness controller 91 controls the target bending stiffness (G+g(t)) based on the change quantity g of the bending stiffness. Accordingly, a bending stiffness distribution suitable for inserting the flexible tube 35 can be obtained; thereby, allowing a safe and simple insertion operation to be implemented, and allowing an easy-to-use endoscope 20 to be provided.

In the present embodiment, the shape calculator 81 determines the change quantity g of the bending stiffness from time displacement of the second insertion path C2 with respect to the first insertion path C1. Therefore, the change quantity g can be easily determined.

The shape calculator 81 determines the change quantity g of the bending stiffness from time integral with respect to the displacement of the second insertion path C2 with respect to the first insertion path C1. Therefore, when the state of displacement, which is a deviation, continues for a longer time, the change quantity g is increased accordingly, so that the bending stiffness can be rapidly approximated to the target bending stiffness (G+g(t)).

The shape calculator 81 determines the change quantity of the bending stiffness from time differential with respect to the displacement of the second insertion path C2 with respect to the first insertion path C1. Therefore, the overshoot can be prevented from occurring, and occurrence of the vibration hunting about the bending stiffness (G+g(t)), which is the target value, can be prevented. Furthermore, in the case where the change quantity g drastically changes, the damping effect would suppress drastic changes in the bending stiffness G.

In the present embodiment, controllability can be improved by time integral and time differential.

The present invention is not limited to the exact embodiment described above; the invention can be embodied by modifying the structural elements without departing from the gist of the invention when being implemented. In addition, various inventions can be made by properly combining the structural elements disclosed in the above embodiment.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A flexible tube insertion apparatus comprising:
a flexible tube that is segmented into segments aligned in a row along a central axis of the flexible tube, and is to be inserted into a insertion target body;
a variable stiffness portion that varies a bending stiffness of the flexible tube by units of the segments;
a state detection sensor that detects state information regarding a state of the flexible tube;
a shape calculator that calculates shape information regarding a shape of the flexible tube based on the state information; and
a stiffness controller that controls a change of the bending stiffness implemented by the variable stiffness portion,
the stiffness controller controlling the change of the bending stiffness implemented by the variable stiffness portion with respect to a segment corresponding to a displacement portion of an insertion path, in accordance with a displacement quantity of a second insertion path of the flexible tube based on the shape information calculated by the shape calculator at a second time, with respect to a first insertion path of the flexible tube based on the shape information calculated by the shape calculator at a first time.

2. The flexible tube insertion apparatus according to claim 1, wherein the shape calculator determines a change quantity of the bending stiffness in accordance with the displacement quantity, and
the stiffness controller controls the bending stiffness based on the change quantity of the bending stiffness.

3. The flexible tube insertion apparatus according to claim 2, wherein the shape calculator determines the change quantity of the bending stiffness from time displacement of the second insertion path with respect to the first insertion path.

4. The flexible tube insertion apparatus according to claim 3, wherein the shape calculator determines the change quantity of the bending stiffness from time integral with respect to the displacement of the second insertion path with respect to the first insertion path.

5. The flexible tube insertion apparatus according to claim 4, wherein the shape calculator determines the change quantity of the bending stiffness from time differential with respect to the displacement of the second insertion path with respect to the first insertion path.
